(19) United States
(12) Patent Application Publication
(10) Pub. No.: US 2023/0358759 A1
(43) Pub. Date: Nov. 9, 2023

(54) CYTOKINE BASED ASSESSMENT OF RECIPIENT ABILITY TO RESPOND TO STEM CELL THERAPY FOR CARTILAGE REGENERATION

(71) Applicant: CREATIVE MEDICAL TECHNOLOGIES, INC., Phoenix, AZ (US)

(72) Inventors: Thomas ICHIM, San Diego, CA (US); Amit PATEL, Salt Lake City, UT (US); Courtney BARTLETT, Niceville, FL (US)

(73) Assignee: CREATIVE MEDICAL TECHNOLOGIES, INC., Phoenix, AZ (US)

(21) Appl. No.: 18/311,445

(22) Filed: May 3, 2023

Related U.S. Application Data
(60) Provisional application No. 63/338,416, filed on May 4, 2022.

(51) Int. Cl. G01N 33/68; A61K 35/28

(52) U.S. Cl. CPC G01N 33/6863; A61K 35/28; G01N 2333/54; G01N 2333/525

(57) ABSTRACT

Disclosed are means of assessing the likelihood of a patient to respond positively to stem cell therapy for cartilage regeneration. In one embodiment enhanced inflammatory cytokines are reduced anti-inflammatory cytokines are assessed in the plasma of a potential patient to assess likelihood of response to therapy. In other embodiments intra-articular or synovial fluid is assessed for levels of cytokines that are detrimental to the success of the stem cell therapy. In other embodiments quality of stem cells in the patient are assessed for cytokine to determine suitability for use in stem cell therapy.

### CROSS-REFERENCE TO RELATED APPLICATIONS

This application claims priority to U.S. Provisional Application No. 63/338,416, titled “Cytokine Based Assessment of Recipient Ability to Respond to Stem Cell Therapy for Cartilage Regeneration” filed May 4, 2022, which is hereby incorporated by reference herein in its entirety.

### FIELD OF THE INVENTION

The invention relates to the use of stem cell therapy to regenerate cartilage in patients in need.

### BACKGROUND

Stem cell therapy has demonstrated potent ability to induce tissue regeneration and healing of tissues which previously were resistant to healing. There exists a great heterogeneity of patient responses to stem cell therapies in which some patients experience significant benefits while others appear to be improved.

There is a need in the art to be able to stratify patients into ones with high and ones with low possibility of response.

Preferred embodiments are directed to methods of assessing possibility of a positive response to stem cell therapy for treatment of cartilage degenerative disease by quantifying levels of pro-inflammatory and anti-inflammatory mediators in systemic circulation and/or local micro-environment.

Preferred methods include embodiments wherein said stem cell therapy is autologous administration of bone marrow aspirate/mononuclear cells.

Preferred methods include embodiments wherein said stem cell therapy is allogeneic administration of bone marrow derived mesenchymal stem cells.

Preferred methods include embodiments wherein said mesenchymal stem cells express CD105.

Preferred methods include embodiments wherein said mesenchymal stem cells express CD90.

Preferred methods include embodiments wherein said mesenchymal stem cells express c-kit.

Preferred methods include embodiments wherein said mesenchymal stem cells express IL-3 receptor.

Preferred methods include embodiments wherein said mesenchymal stem cells are autologous.

Preferred methods include embodiments wherein assessment of plasma levels of interleukin-1 beta is performed as an indicator of patient inflammatory status.

Preferred methods include embodiments wherein said interleukin-1 beta is assessed by ELISA.

Preferred methods include embodiments wherein plasma is treated with a stabilizing agent subsequently to blood collection in order to enhance stability of said interleukin-1 beta.

Preferred methods include embodiments wherein said stabilizing agent is albumin.

Preferred methods include embodiments wherein said stabilizing agent is a protease inhibitor.

Preferred methods include embodiments wherein said stabilizing agent is a matrix metallo-protease inhibitor.

Preferred methods include embodiments wherein assessment of plasma levels of interleukin-6 is performed as an indicator of patient inflammatory status.

Preferred methods include embodiments wherein said interleukin-6 is assessed by ELISA.

Preferred methods include embodiments wherein plasma is treated with a stabilizing agent subsequently to blood collection in order to enhance stability of said interleukin-6.

Preferred methods include embodiments wherein said stabilizing agent is albumin.

Preferred methods include embodiments wherein said stabilizing agent is a protease inhibitor.

Preferred methods include embodiments wherein said stabilizing agent is a matrix metallo-protease inhibitor.

Preferred methods include embodiments wherein assessment of plasma levels of interleukin-6 is performed as an indicator of patient inflammatory status.

Preferred methods include embodiments wherein said interleukin-6 is assessed by ELISA.

Preferred methods include embodiments wherein plasma is treated with a stabilizing agent subsequently to blood collection in order to enhance stability of said interleukin-6.

Preferred methods include embodiments wherein said stabilizing agent is albumin.

Preferred methods include embodiments wherein said stabilizing agent is a protease inhibitor.

Preferred methods include embodiments wherein said stabilizing agent is a matrix metallo-protease inhibitor.

Preferred methods include embodiments wherein assessment of plasma levels of interleukin-18 is performed as an indicator of patient inflammatory status.

Preferred methods include embodiments wherein said interleukin-18 is assessed by ELISA.

Preferred methods include embodiments wherein plasma is treated with a stabilizing agent subsequently to blood collection in order to enhance stability of said interleukin-18.

Preferred methods include embodiments wherein said stabilizing agent is albumin.

Preferred methods include embodiments wherein said stabilizing agent is a protease inhibitor.

Preferred methods include embodiments wherein said stabilizing agent is a matrix metallo-protease inhibitor.

Preferred methods include embodiments wherein said stem cell therapy is administration of bone marrow concentrate.

Preferred methods include embodiments wherein said stem cell therapy is administration of bone marrow mononuclear cells.

### DETAILED DESCRIPTION OF THE INVENTION

The current invention provides cytokine-based tests for assessing ability of the patient to respond to stem cell therapy. The foundation of these tests are the signals generated by the body as a result of various underlying conditions that accumulate in the body possessing higher or lower ability to response to cellular therapies. The invention focuses on treatment of articular injuries and osteoarthritis as defined by cartilage erosion. The technology provides methods for assessing the inflammatory and anti-inflammatory cytokines derived from biological materials. Such methods may be used in a variety of ways in designing and producing devices and compositions and in clinical practice, so as to measure, control, improve, assess quality, and otherwise manage the production of biologically-derived anti-inflammatory compositions and their use in treating disorders associated with inflammatory cytokines. For example, such methods may be used to assess devices used in making such anti-inflammatory compositions, methods for generating such compositions, and in methods for determining whether an anti-inflammatory composition is suitable for treating an inflammatory disorder.

Disclosed herein are inventive assay methods comprising measuring the levels of one or more cytokines in the sample in a patient being considered for stem cell or regenerative therapy, wherein said patient is suffering from osteoarthritis or other cartilage degenerating disease. In some embodiments of the invention, the method may involve determining from measured cytokine levels if the patient has an inflammatory disease and/or determining from measured cytokine levels, the level of inflammation due to an inflammatory disease and/or obtaining and measuring samples at different times to monitor the progression of an inflammatory disease or the effectiveness of treatments for such disease. In other embodiments the invention provides means of identifying patients whose bone marrow would support the use for cartilage regeneration. In one embodiment, the method includes measuring the level of cytokines such as those associated with inflammation. Cytokines that may be assayed for the purpose of the current invention include:
1. Interleukin-1 beta (IL-1β) is released primarily by monocytes and macrophages as well as by nonimmune cells, such as fibroblasts and endothelial cells, during cell injury, infection, invasion, and inflammation. Very recently, it was found that IL-1β is expressed in nociceptive DRG neurons. IL-1β expression is enhanced following crush injury to peripheral nerve and after trauma in microglia and astrocytes in the central nervous system (CNS). IL-1β can produce hyperalgesia following either intraperitoneal, intracerebroventricular or intraplantar injection. Moreover, IL-1β was found to increase the production of substance P and prostaglandin E2 (PGE2) in a number of neuronal and glial cells. IL-1ra, a specific IL-1 receptor antagonist, competitively binds to the same receptor as IL-1β but does not transduce a cellular signal, thereby blocking IL-1β-mediated cellular changes. Administrations of IL-1ra and other anti-inflammatory cytokines have been demonstrated to prevent or attenuate cytokine-mediated inflammatory hyperalgesia and nerve-injury induced mechanical allodynia. Cytokines belonging to the interleukin-1 beta family include: IL18, IL18BP, IL1A, IL1B, IL1F10, IL1F3/IL1RA, IL1F5, IL1F6, IL1F7, IL1F8, IL1RL2, IL1F9, and IL33.
2. Interleukin-6 (IL-6) has been shown to play a central role in the neuronal reaction to nerve injury. Suppression of IL-6R by in vivo application of anti-IL-6R antibodies led to reduced regenerative effects. IL-6 is also involved in microglial and astrocytic activation as well as in regulation of neuronal neuropeptides expression. There is evidence that IL-6 contributes to the development of neuropathic pain behavior following a peripheral nerve injury. For example, sciatic cryoneurolysis, a sympathetically-independent model of neuropathic pain involving repeatedly freezing and thawing a section of the sciatic nerve, results in increased IL-6 immunoreactivity in the spinal cord. In addition, intrathecal infusion of IL-6 induces tactile allodynia and thermal hyperalgesia in intact and nerve-injured rats, respectively. Cytokines belonging to the IL-6 family include:
3. Tumor Necrosis Factor TNF-α, also known as cachectin, is another inflammatory cytokine that plays a well-established, key role in some pain models. TNF acts on several different signaling pathways through two cell surface receptors, TNFR1 and TNFR2 to regulate apoptotic pathways, NF-kB activation of inflammation, and activate stress-activated protein kinases (SAPKs). TNF-α receptors are present in both neurons and glia. TNF-α has been shown to play important roles in both inflammatory and neuropathic hyperalgesia. Intraplantar injection of complete Freund's adjuvant in adult rats resulted in significant elevation in the levels of TNF-α, IL-1β, and nerve growth factor (NGF) in the inflamed paw. A single injection of anti-TNF-α antiserum before the CFA significantly delayed the onset of the resultant inflammatory hyperalgesia and reduced IL-1β but not NGF levels. Intraplantar injection of TNF-α also produces mechanical and thermal hyperalgesia. It has been found that TNF-α injected into nerves induces Wallerian degeneration and generates the transient display of behaviors and endoneurial pathologies found in experimentally painful nerve injury. TNF binding protein (TNF-BP), an inhibitor of TNF, is a soluble form of a transmembrane TNF-receptor. When TNF-BP is administered systemically, the hyperalgesia normally observed after lipopolysaccharide (LPS) administration is completely eliminated. Intrathecal administration of a combination of TNF-BP and IL-1 antagonist attenuated mechanical allodynia in rats with L5 spinal nerve transection. Members of the TNF alpha family include: BAFF, 4-1BBL, TNFSF8, CD40LG, CD70, CD95L/CD178, EDA-A1, TNFSF14, LTA/TNFB, LTB, TNFa, TNFSF10, TNFSF11, TNFSF12, TNFSF13, TNFSF15
4. Chemokines. A variety of cytokines are known to induce chemotaxis. One particular subgroup of structurally related cytokines is known as chemokines. The term chemotactic cytokines (CHEMOtactic CytoKINES) usually refers to this. These factors represent a family of low molecular weight secreted proteins that primarily function in the activation and migration of leukocytes although some of them also possess a variety of other functions. Chemokines have conserved cysteine residues that allow them to be assigned to four groups: C—C chemokines (RANTES, monocyte chemoattractant protein or MCP-1, monocyte inflammatory protein or MIP-1α, and MIP-1β), C—X—C chemokines (IL-8 also called growth related oncogene or GRO/KC), C chemokines (lymphotactin), and CXXXC chemokines (fractalkine). Various chemokines including MIP-1α, MCP-1 and GRO/KC are up-regulated not only in models of neuroinflammatory and demylinating diseases, but also in various forms of CNS trauma and in injured peripheral nerve. Receptors for MCP-1, MIP-1α and GRO/KC are expressed on DRG neurons. Interestingly, mice lacking the CCR2 receptor completely fail to develop mechanical allodynia in the partial sciatic injury model although pain sensitivity in uninjured animals is normal. In the same study, normal mice showed a sustained upregulation of the receptors in both DRG and peripheral nerve after the injury. This suggests that the chemokines, including MCP-1 in particular, play very key roles in neuropathic pain as well as in neuroinflammatory conditions.

In certain embodiments, the method includes measuring a plurality of cytokines and may also include comparing the levels of these cytokines to cytokine profiles determined to be indicative of the disease. A variety of samples may be analyzed. In certain embodiments, the samples may be obtained by a non-surgically invasive procedure from a human patient and may, for example, include blood, serum, plasma, fecal, or urine samples.

In one embodiment of the invention, cytokines are assayed by ELISA before administration of stem cells. These cytokines may be selected from may be selected from a group comprising IL-1.beta., IL-12p70, IL-10, IL-2, GM-CSF, TNF, IL-8, IL-4, IL-5, IL-6, Eotaxin, IFN-.alpha., IFN-.gamma., sIL-6R, IL-12 (total), IL-13, MIP-1.beta., MCP-1, RANTES and sTNFRII.

In one specific example, the cytokines are selected from the group consisting of IL-12p70, IL-10, IL-2, TNF, IL-8, IL-4, IL-5, IL-6, Eotaxin, sIL-6R, IL-12 (total), MIP-1.beta., MCP-1, RANTES and sTNFRII. In another specific example, the cytokines are selected from the group consisting of Eotaxin, sIL-6R, MIP-1.beta., MCP-1, and RANTES. In another specific example, the first cytokine is MCP-1 and the additional cytokine is MIP-1.beta.

In one embodiment of the invention, sTNFRII is selected as the first cytokine. Thus, the invention is a method for diagnosing responsiveness to stem cell therapy comprising: measuring the sTNFRII level in a sample, for example, a sample obtained from a patient and assessment is made of one or more additional cytokines (e.g., levels of one or more of IL-1.beta., IL-12p70, IL-10, IL-2, GM-CSF, TNF, IL-8, IL-4, IL-5, IL-6, Eotaxin, IFN-.alpha., IFN-.gamma., sIL-6R, IL-12 (total), IL-13, MIP-1.beta., MCP-1 and/or RANTES); and diagnosing from the sTNFRII level and from one or more additional cytokine level. In one specific example, the additional cytokine(s) are selected from the group consisting of one or more of IL-12p70, IL-10, IL-2, TNF, IL-8, IL-4, IL-5, IL-6, Eotaxin, sIL-6R, IL-12 (total), MIP-1.beta., MCP-1 and RANTES. In another specific example, the additional cytokines are selected from the group consisting of one or more of Eotaxin, sIL-6R, MIP-1.beta., MCP-1, and RANTES. In another specific example, the additional cytokine is MIP-1.beta.

Determining from the IL-33 level and from one or more additional cytokine levels if the patient has potential for stem cell therapy may comprise comparing the IL-33level and one or more additional levels to a cytokine profile determined to be indicative of stem cell responsiveness. The step of comparing may comprise comparing cytokine levels to detection cut-off values, comparing ratios of levels to detection cut-off ratio values and/or comparing levels to detection cut-off lines, curves or surfaces in multi-analyte correlation plots. In one embodiment, a IL-33level above a IL-33detection cut-off value and a level of an additional cytokine below a cytokine detection cut-off value are considered indicative of stem cell response. In another embodiment, a ratio of the IL-33level to one additional cytokine level above a detection cut-off ratio value is considered indicative of non response to stem cells. In yet another embodiment, a IL-33level above a IL-33detection cut-off line is considered indicative of response to stem cell therapy. In yet another embodiment IL-6 is selected as a first cytokine and IL-13 as a second cytokine.

One specific example of the invention also relates to a method for selecting patients suitable for stem cell administration for osteoarthritis by measuring pair-wise cytokine level profiles selected from the group consisting of IL-33/RANTES, IL-33/sIL-6R, sIL-6R/RANTES, IL-5/sIL-6R and L-33/IL-4.

In one embodiment of the invention, a method is disclosed that is useful for distinguishing patients that have a higher possibility of responding to stem cell therapy compared to patients that have a lower possibility of responding to stem cell therapy on the basis of a measured IL-33 level and one or more additional measured cytokine levels. In one example, the IL-33 levels and one or more additional cytokine levels are blood, serum or plasma levels.

For example, patients potentially being able to respond to stem cell therapy can be distinguished from patients with a low possibility of responding, according to the invention, by comparing the IL-33 level and one or more additional cytokine levels to profiles determined to be indicative of inflammatory status. The step of comparing may comprise comparing levels to discrimination cut-off values, comparing ratios of levels to discrimination cut-off ratio values, and/or comparing levels to discrimination cut-off lines. In one embodiment, potential responding patients are distinguished from non-responding patients by comparing the IL-33 level to a IL-33 discrimination cut-off value, wherein a IL-33 level below said IL-33 discrimination cut-off value is considered indicative of increased possibility of patient responding and above the IL-33 discrimination cut-off value is considered indicative of low possibility.

In another embodiment, patients potentially being able to respond to stem cell therapy can be distinguished from patients with a low possibility of responding, according to the invention, by comparing the IL-1 beta level and one or more additional cytokine levels to profiles determined to be indicative of inflammatory status. The step of comparing may comprise comparing levels to discrimination cut-off values, comparing ratios of levels to discrimination cut-off ratio values, and/or comparing levels to discrimination cut-off lines. In one embodiment, potential responding patients are distinguished from non-responding patients by comparing the IL-1 beta level to a IL-1 beta discrimination cut-off value, wherein a IL-1 beta level below said IL-1 beta discrimination cut-off value is considered indicative of increased possibility of patient responding and above the IL-1 beta discrimination cut-off value is considered indicative of low possibility.

In another embodiment, patients potentially being able to respond to stem cell therapy can be distinguished from patients with a low possibility of responding, according to the invention, by comparing the IL-6 level and one or more additional cytokine levels to profiles determined to be indicative of inflammatory status. The step of comparing may comprise comparing levels to discrimination cut-off values, comparing ratios of levels to discrimination cut-off ratio values, and/or comparing levels to discrimination cut-off lines. In one embodiment, potential responding patients are distinguished from non-responding patients by comparing the IL-6 level to a IL-6 discrimination cut-off value, wherein a IL-6 level below said IL-6 discrimination cut-off value is considered indicative of increased possibility of patient responding and above the IL-6 discrimination cut-off value is considered indicative of low possibility.

In another embodiment, patients with a higher probability of responses are distinguished from patients with a lower probability of responses by comparing the IL-1 beta level to a IL-1 beta discrimination cut-off line, wherein IL-1 beta level below the IL-1 beta discrimination cut-off line is considered indicative of higher probability of response to stem cell therapy and above the IL-1 beta discrimination cut-off line is considered indicative of lower probability of response.

In another embodiment, patients with a higher probability of responses are distinguished from patients with a lower probability of responses by comparing the IL-33 level to a IL-33 discrimination cut-off line, wherein IL-33 level below the IL-33 discrimination cut-off line is considered indicative of higher probability of response to stem cell therapy and above the IL-33 discrimination cut-off line is considered indicative of lower probability of response.

In another embodiment, patients with a higher probability of responses are distinguished from patients with a lower probability of responses by comparing a measured IL-1 beta level to a cytokine profile defined as areas situated between a first detection cut-off line and a second discrimination cut-off line on a correlation plot.

In another embodiment, patients with a higher probability of responses are distinguished from patients with a lower probability of responses by comparing a measured IL-6 level to a cytokine profile defined as areas situated between a first detection cut-off line and a second discrimination cut-off line on a correlation plot.

In another embodiment, patients with a higher probability of responses are distinguished from patients with a lower probability of responses by comparing a measured IL-8 level to a cytokine profile defined as areas situated between a first detection cut-off line and a second discrimination cut-off line on a correlation plot.

In another embodiment, patients with a higher probability of responses are distinguished from patients with a lower probability of responses by comparing a measured IL-9 level to a cytokine profile defined as areas situated between a first detection cut-off line and a second discrimination cut-off line on a correlation plot.

In another embodiment, patients with a higher probability of responses are distinguished from patients with a lower probability of responses by comparing a measured IL-33 level to a cytokine profile defined as areas situated between a first detection cut-off line and a second discrimination cut-off line on a correlation plot.

In one embodiment, patients with a higher probability of responses are distinguished from patients with a lower probability of responses by comparing two or more cytokines measured in a patient to a profile of these two or more cytokines, e.g., values, ratios, lines or zones on the correlation plot, indicative of a patient having higher possibility of responding to therapy or lower possibility. In one specific example, pare-wise cytokine profiles are selected from the group consisting of, but not limited to, sTNFRII/RANTES, sTNFRII/sIL-6R, and sTNFRII/IL-4. Another embodiment of the invention relates to methods for measuring the extent of inflammation associated with osteoarthritis in the patient. The inventive methods may include an assay method comprising: measuring the level of IL-17 in a sample, for example, a sample obtained from a patient that has or is suspected to have an inflammatory disease; and determining from the level of IL-17 the extent of inflammation from the disease.

One embodiment of the invention includes a method comprising: measuring a level of a first cytokine, for example, measuring in a sample obtained from a patient that has or is suspected to have an inflammatory disease such as osteoarthritis; measuring the level of one or more additional cytokines, wherein the one or more additional cytokines differ from the first cytokine; and determining from measured levels the extent of inflammation from the disease. In one embodiment, the cytokines comprise one or more cytokines selected from the group consisting of IL-1.beta., IL-12p70, IL-10, IL-2, GM-CSF, TNF, IL-8, IL-4, IL-5, IL-6, Eotaxin, IFN-.alpha., IFN-.gamma., sIL-6R, IL-12 (total), IL-13, MIP-1.beta., MCP-1, RANTES and sTNFRII. In another embodiment, the cytokines are selected from the group consisting of IL-12p70, IL-10, IL-2, TNF, IL-8, IL-4, IL-5, IL-6, Eotaxin, sIL-6R, IL-12 (total), MIP-1.beta., MCP-1, RANTES and sTNFRII. In another embodiment, the cytokines comprise one or more cytokines selected from the group consisting of Eotaxin, sIL-6R, MIP-1.beta., MCP-1, and RANTES. In another embodiment, the first cytokine is MCP-1 and the second cytokine is MIP-1.beta. In another embodiment, the first cytokine is sTNFRII. In one specific example of this embodiment, the additional cytokine(s) are selected from the group consisting of IL-12p70, IL-10, IL-2, TNF, IL-8, IL-4, IL-5, IL-6, Eotaxin, sIL-6R, IL-12 (total), MIP-1.beta., MCP-1 and RANTES. In another specific example, the additional cytokine(s) are selected from the group consisting of Eotaxin, sIL-6R, MIP-1.beta., MCP-1, and RANTES. In another specific example, the additional cytokine is MIP-1.beta. In another specific example, a pair of cytokines is selected from the group consisting of, but not limited to, sTNFRII/RANTES, sTNFRII/sIL-6R, and sTNFRII/IL-4.

In one embodiment, the invention teaches methods of monitoring the progression or treatment of inflammatory conditions such as osteoarthritis. The invention includes a method for monitoring the progression or treatment inflammatory conditions such as osteoarthritis comprising: measuring the levels of IL-17 in samples obtained at different times, for example, samples obtained from a patient that has or is suspected to have an inflammatory disease; and determining from the levels of IL-17 on the progression or efficacy of treatment of the disease. In one embodiment the treatment intervention is administration of stem cells.

In one embodiment of the invention, methods for monitoring the progression or treatment of an inflammatory disease such as osteoarthritis are disclosed, said methods comprising: measuring the levels of a first cytokine in samples obtained at different times, for example, samples obtained from a patient that has or is suspected to have an inflammatory disease; measuring the levels of one or more additional cytokines in the samples from the same patient obtained at the same times as samples for the first cytokine, for example, the same samples, wherein the one or more additional cytokines differ from the first cytokine; and determining from measured levels the progression or efficacy of treatment of the disease. In one embodiment, the cytokines comprise one or more cytokines selected from the group consisting of IL-1.beta., IL-12p70, IL-10, IL-2, GM-CSF, TNF, IL-8, IL-4, IL-5, IL-6, Eotaxin, IFN-.alpha., IFN-.gamma., sIL-6R, IL-12 (total), IL-13, MIP-1.beta., MCP-1, RANTES and sTNFRII. In another embodiment, the cytokines comprise one or more cytokines selected from the group consisting of IL-12p70, IL-10, IL-2, TNF, IL-8, IL-4, IL-5, IL-6, Eotaxin, sIL-6R, IL-12 (total), MIP-1.beta., MCP-1, RANTES and sTNFRII. In another embodiment, the cytokines comprise one or more cytokines selected from the group consisting of Eotaxin, sIL-6R, MIP-1.beta., MCP-1, and RANTES. In another embodiment, the first cytokine is MCP-1 and the second cytokine is MIP-1.beta. In one embodiment the treatment intervention is stem cell therapy.

In another embodiment of the invention cytokines are assessed as part of a means of determining suitability of patients to receive stem cell therapy. Accordingly, in this embodiment, the first cytokine is IL-33. In one specific example of this embodiment, the additional cytokine(s) are selected from the group consisting of IL-12p70, IL-10, IL-2, IL-17, IL-18, TNF, IL-8, IL-4, IL-5, IL-6, Eotaxin, sIL-6R, IL-12 (total), MIP-1.beta., MCP-1 and RANTES. In another specific example, the additional cytokine(s) are selected from the group consisting of Eotaxin, sIL-6R, MIP-1.beta., MCP-1, and RANTES. In another specific example, the additional cytokine is MIP-1.beta. In another specific example, a pair of cytokines is selected from the group consisting of, but not limited to, IL-33/RANTES, IL-33/sIL-6R, and sTNFRII/IL-4.

Another aspect of the invention involves a method for evaluation of the effectiveness of a stem cell or stem cell candidate for treating inflammation and/or osteoarthritis. For example, the invention includes a method for evaluating the effectiveness of a drug and/or drug candidate comprising: exposing a human or non-human animal with osteoarthritis and/or a model system, for example, a tissue, cell culture or a biochemical system, to the stem cell candidate; measuring the levels of IL-33 in a sample obtained from the human or non-human animal or a model system; and determining from the level the effectiveness of the drug or drug candidate.

In another embodiment, the cytokines comprise one or more cytokines selected from the group consisting of Eotaxin, sIL-6R, MIP-1.beta, MCP-1, and RANTES. In another embodiment, the first cytokine is MCP-1 and the second cytokine is MIP-1.beta. In yet another embodiment, the first cytokine is sTNFRII. In one specific example of this embodiment, the additional cytokine(s) are selected from the group consisting of IL-12p70, IL-10, IL-2, TNF, IL-8, IL-4, IL-5, IL-6, Eotaxin, sIL-6R, IL-12 (total), MIP-1.beta, MCP-1 and RANTES. In another specific example, the additional cytokine(s) are selected from the group consisting of Eotaxin, sIL-6R, MIP-1.beta, MCP-1, and RANTES. In another specific example, the additional cytokine is MIP-1.beta. In another specific example, a pair of cytokines is selected from the group consisting of, but not limited to, sTNFRII/RANTES, sTNFRII/sIL-6R, and sTNFRII/IL-4.

Disclosed herein are inventive methods for conducting diagnostic tests for the detection of inflammatory diseases such as inflammatory bowel disease (IBD). The diagnostic tests may comprise measuring analytes in biological samples, for example measuring disease markers, markers of inflammation, and/or cytokines, where the levels of the analytes are indicative of the presence or severity of an inflammatory disease. One aspect of the invention is identifying diagnostically valuable markers of IBDs, for example diagnostically valuable markers of Crohn's disease (CD) and ulcerative colitis (UC). Another aspect of the invention relates to methods for detecting and/or distinguishing various IBDs, such as CD and UC. Another aspect of the invention further relates to methods for monitoring the progression or treatment of inflammatory bowel disease in a patient by administering and/or repetitively administering the diagnostic tests according to the methods of the present invention. In one example, the diagnostic methods may be used to evaluate the effectiveness of a drug or drug candidate for treating inflammatory diseases by measuring the effect of the drug or drug candidate on the levels of disease-specific analytes in samples from patients, animal models, tissue samples and cell cultures treated with a drug or a drug candidate. Another aspect of the invention provides methods for determining the efficacy of particular candidate analytes, such as particular cytokine(s), for acting as diagnostic marker(s) in the inventive methods for the diagnosis and/or monitoring of inflammatory bowel disease and for screening drugs or drug candidates for efficacy in treating inflammatory bowel disease.

The invention provides the assessment of various substances associated with inflammation or suppression of inflammation as means of quantifying risk of stem cell therapy failure in conditions such as osteoarthritis treated with stem cells. In some embodiments stem cells are derived from the bone marrow which is unmanipulated, in other embodiments, stem cells are cellular populations that have been expanded ex vivo. Ex vivo expanded stem cells can be used allogeneically or in an autologous manner. Mesenchymal stem cells have been cleared by regulators in certain conditions. In some embodiments, analytes may be measured using the assay methods of the present invention which include inflammatory markers, such as cytokines, secreted proteins that are involved in regulation of immune response. Cytokines under the current invention include interleukins (ILs), interferons (IFNs), chemokines, tumor necrosis factors (TNFs), and a variety of colony stimulating factors (CSFs). The term cytokines, as used herein, also includes soluble cytokine receptors. Specific cytokines that may be measured in the assays of the invention include, but are not limited to, cytokines linked to TH1 response, cytokines linked to TH2 response, pro-inflammatory cytokines and/or cytokines selected from the group consisting of IL-1.beta, IL-12p70, IL-10, IL-2, granulocyte-macrophage colony stimulating factor (GM-CSF), TNF-.alpha., IL-8, IL-4, IL-5, IL-6, Eotaxin, IFN-.alpha., IFN-.gamma., soluble IL-6 receptor (sIL-6R), IL-12 (total), IL-13, MIP-1.beta, MCP-1, RANTES and soluble TNF-.beta. receptor II (sTNFRII. According to one aspect of the invention, the levels of cytokine or other disease marker candidates are measured in the samples collected from individuals suffering from osteoarthritis and from healthy individuals. Within non-limiting examples of this invention, specific cytokines valuable as a marker for distinguishing between normal and diseased patients could be identified using visual inspection of the data, for example, data plotted on a one-dimensional or multi-dimensional graph, or by using methods of statistical analysis, such as a statistically weighted difference between control individuals and diseased patients and/or Receiver Operating Characteristic (ROC) curve analysis. For example in one exemplary embodiment of the present invention, diagnostically valuable cytokines may be first identified using a statistically weighted difference between control individuals and diseased patients, calculated as

It is known that someone of ordinary skill in the art of diagnostic assays and statistical analysis of data, given the teaching and guidance provided herein, will be able to select without undue burden appropriate cut-off values, lines, ratios, zones etc. for best meeting the needs (e.g., sensitivity and specificity) for a particular application. A variety of statistical tools, such as, for example, receiver operating characteristic (ROC) curves, are available for evaluating the effect of adjustments to cut-offs on assay performance (e.g., predicted true positive fraction, false positive fraction, true negative fraction and false negative fraction). Alternatively, statistical analysis of patient populations can allow conversion of specific analyte values into probabilities that the patient has or does not have a disease. For background on the selection and analysis of populations of individuals so as to determine reference ranges see Boyd J. C. “Reference Limits in the Clinical Laboratory” in Professional Practice in Clinical Chemistry: A Companion Text; D. R. Dufour Ed., 1999, Washington D.C.: American Assoc. Clin. Chem., Chapter 2, pp. 2-1 to 2-7. For background on the selection of decision limits (i.e., cut-offs) or the calculation, from test results, of disease likelihood see Boyd J. C. “Statistical Aids for Test Interpretation” in Professional Practice in Clinical Chemistry: A Companion Text; D. R. Dufour Ed., 1999, Washington D.C.: American Assoc. Clin. Chem., Chapter 3, pp. 3-1 to 3-11. The inventors attest that given the teachings of the present invention, a skilled artisan will also recognize that the choice of first cytokine and one or more additional cytokines may transpose correlation plot axes and consequently the criteria for determining whether measured cytokine levels of a patient's samples falling above or below particular cut-off ratios, lines and/or profiles is indicative of a disease state and will be able to adjust the analysis accordingly. For the practice of the invention, the cytokine levels may be measured using any of a number of techniques available to the person of ordinary skill in the art, e.g., direct physical measurements (e.g., mass spectrometry) or binding assays (e.g., immunoassays, agglutination assays, and immunochromatographic assays). The method may also comprise measuring a signal that results from a chemical reaction, e.g., a change in optical absorbance, a change in fluorescence, the generation of chemiluminescence or electrochemiluminescence, a change in reflectivity, refractive index or light scattering, the accumulation or release of detectable labels from the surface, the oxidation or reduction or redox species, an electrical current or potential, changes in magnetic fields, etc. Suitable detection techniques may detect binding events by measuring the participation of labeled binding reagents through the measurement of the labels via their photoluminescence (e.g., via measurement of fluorescence, time-resolved fluorescence, evanescent wave fluorescence, up-converting phosphors, multi-photon fluorescence, etc.), chemiluminescence, electrochemiluminescence, light scattering, optical absorbance, radioactivity, magnetic fields, enzymatic activity (e.g., by measuring enzyme activity through enzymatic reactions that cause changes in optical absorbance or fluorescence or cause the emission of chemiluminescence). Alternatively, detection techniques may be used that do not require the use of labels, e.g., techniques based on measuring mass (e.g., surface acoustic wave measurements), refractive index (e.g., surface plasmon resonance measurements), or the inherent luminescence of an analyte. Going into some level of detail, binding assays for measuring cytokine levels may use solid phase or homogenous formats. Suitable assay methods include sandwich or competitive binding assays. Examples of sandwich immunoassays are described in U.S. Pat. No. 4,168,146 to Grubb et al. and U.S. Pat. No. 4,366,241 to Tom et al., both of which are incorporated herein by reference. Examples of competitive immunoassays include those disclosed in U.S. Pat. No. 4,235,601 to Deutsch et al., U.S. Pat. No. 4,442,204 to Liotta, and U.S. Pat. No. 5,208,535 to Buechler et al., all of which are incorporated herein by reference. It is being disclosed as a given that multiple cytokines may be measured using a multiplexed assay format, e.g., multiplexing through the use of binding reagent arrays, multiplexing using spectral discrimination of labels, multiplexing by flow cytometric analysis of binding assays carried out on particles (e.g., using the Luminex system). Suitable multiplexing methods include array based binding assays using patterned arrays of immobilized antibodies directed against the cytokines of interest. Various approaches for conducting multiplexed assays have been described. For example, multiplexed testing is described in U.S. patent application Ser. Nos. 10/185,274 and 10/185,363, both filed on Jun. 28, 2002, entitled “Assay Plates, Reader Systems and Methods For Luminescence Test Measurements,” published as U.S. Pat. Publ. No. 20040022677 and US20050052646, respectively, U.S. patent application Ser. No. 10/238,960, filed Sep. 10, 2002, entitled “Methods, Reagents, Kits and Apparatus for Protein Function,” published as U.S. Pat. Publ. No. 20030207290, U.S. patent application Ser. No. 10/238,391, filed Sep. 10, 2002, entitled “Methods and apparatus for conducting multiple measurements on a sample”; published as U.S. Pat. Publ. No. 20030113713, U.S. patent application Ser. No. 10/980,198, filed on Nov. 3, 2004, entitled “Modular Assay Plates, Reader System and Methods For Test Measurements,” published as U.S. Pat. Publ. No. 20050142033; and U.S. patent application Ser. No. 10/744,726, filed on Dec. 23, 2003, entitled “Assay Cartridges and Methods of Using Same,” published as U.S. Pat. Publ. No. 20040189311, each of which is incorporated by this reference. One approach to multiplexing binding assays involves the use of patterned arrays of binding reagents (see, e.g., U.S. Pat. Nos. 5,807,522 and 6,110,426, both entitled “Methods for Fabricating Microarrays of Biological Samples” issued Sep. 15, 1998 and Aug. 29, 2000 respectively, Delehanty J B, Printing functional protein microarrays using piezoelectric capillaries, Methods Mol Biol. (2004) 278:135-44; Lue R Y, Chen G Y, Zhu Q, Lesaicherre M L, Yao S Q, Site-specific immobilization of biotinylated proteins for protein microarray analysis, Methods Mol Biol. (2004) 278:85-100; Lovett, Toxicogenomics: Toxicologists Brace for Genomics Revolution, Science (2000) 289: 536-537; Berns A., Cancer: Gene expression in diagnosis, Nature (2000) 403, 491-492; Walt, Molecular Biology: Bead-based Fiber-Optic Arrays, Science (2000) 287: 451-452 for more details). Another approach involves the use of binding reagents coated on beads that can be individually identified and interrogated. International Patent publication WO9926067A1 (Watkins et al.) describes the use of magnetic particles that vary in size to assay multiple analytes; particles belonging to different distinct size ranges are used to assay different analytes. The particles are designed to be distinguished and individually interrogated by flow cytometry. Vignali has described a multiplex binding assay in which 64 different bead sets of microparticles are employed, each having a uniform and distinct proportion of two dyes (Vignali, D. A. A., “Multiplexed Particle-Based Flow Cytometric Assays,” J. Immunol. Meth. (2000) 243:243-255). A similar approach involving a set of 15 different beads of differing size and fluorescence has been disclosed as useful for simultaneous typing of multiple pneumococcal serotypes (Park, M. K. et al., “A Latex Bead-Based Flow Cytometric Immunoassay Capable Of Simultaneous Typing Of Multiple Pneumococcal Serotypes (Multibead Assay),” Clin Diagn Lab Immunol. (2000) 7:486-9). Bishop, J. E. et al. have described a multiplex sandwich assay for simultaneous quantification of six human cytokines (Bishop, J. E. et al., “Simultaneous Quantification of Six Human Cytokines in a Single Sample Using Microparticle-based Flow Cytometric Technology,” Clin Chem. (1999) 45:1693-1694).

The inventors present that in various embodiments of the invention, tests may be conducted on a single sample including, but not limited to, blood, serum, plasma, hair, sweat, urine, feces, tissue, biopsies, saliva, skin, mucosa, CNS fluid, bone marrow, tissue extracts, cells, cell extracts, cell culture supernatants, and lymphatic fluids. Particularly advantageous are blood, blood serum, blood plasma, fecal matter, biopsy tissue, intestinal mucosa and urine. Specifically advantageous are blood, blood serum, blood plasma, fecal and urine samples due to the easy and non-surgically invasive collection techniques.

Within the practice of the invention, and utilizing techniques accepted in the art, a diagnostic test may also be is conducted in a single assay chamber, such as a single well of an assay plate or an assay chamber that is an assay chamber of a cartridge. The assay modules (for example assay plates or cartridges, or multi-well assay plates), methods and apparatuses for conducting assay measurements suitable for the present invention are described, for example, in U.S. patent application Ser. Nos. 10/185,274 and 10/185,363, both filed on Jun. 28, 2002, entitled “Assay Plates, Reader Systems and Methods For Luminescence Test Measurements,” published as U.S. Pat. Publ. No. 20040022677 and US20050052646, respectively, U.S. patent application Ser. No. 10/980,198, filed on Nov. 3, 2004, entitled “Modular Assay Plates, Reader System and Methods For Test Measurements,” published as U.S. Pat. Publ. No. 20050142033, and U.S. patent application Ser. No. 10/744,726, filed on Dec. 23, 2003, entitled “Assay Cartridges and Methods of Using Same,” published as U.S. Pat. Publ. No. 20040189311, each of which is incorporated by this reference. Assay plates and plate readers are now commercially available (MULTI-SPOT® and MULTI-ARRAY™ plates and SECTOR™ instruments, Meso Scale Discovery, a division of Meso Scale Diagnostics, LLC, Gaithersburg, Md.). Various diagnostic tests of the present invention may be further supplemented with a diagnostic test to determine if the patient has viral or bacterial infection. Thus, in certain embodiments, the invention further comprises determining if the patient has viral or bacterial infection. Various diagnostic tests of the present invention may be further supplemented with visual patient observation by the doctor, radiological testing and/or histological testing of the patient. The methods of the invention may further comprise administering to the tested patient an effective amount of drug for effective treatment of osteoarthritis and/or other inflammatory diseases.

1. A method of assessing possibility of a positive response to stem cell therapy for treatment of cartilage degenerative disease by quantifying levels of pro-inflammatory and anti-inflammatory mediators in systemic circulation and/or local micro-environment.

2. The method of claim 1, wherein said stem cell therapy is autologous administration of bone marrow aspirate/mononuclear cells.

3. The method of claim 1, wherein said stem cell therapy is allogeneic administration of bone marrow derived mesenchymal stem cells.

4. The method of claim 1, wherein assessment of plasma levels of interleukin-1 beta is performed as an indicator of patient inflammatory status.

5. The method of claim 4, wherein said interleukin-1 beta is assessed by ELISA.

6. The method of claim 4, wherein plasma is treated with a stabilizing agent subsequently to blood collection in order to enhance stability of said interleukin-1 beta.

7. The method of claim 6, wherein said stabilizing agent is albumin.

8. The method of claim 6, wherein said stabilizing agent is a protease inhibitor.

9. The method of claim 6, wherein said stabilizing agent is a matrix metallo-protease inhibitor.

10. The method of claim 1, wherein assessment of plasma levels of interleukin-6 is performed as an indicator of patient inflammatory status.

11. The method of claim 10, wherein said interleukin-6 is assessed by ELISA.

12. The method of claim 1, wherein assessment of plasma levels of interleukin-18 is performed as an indicator of patient inflammatory status.

13. The method of claim 12, wherein said interleukin-18 is assessed by ELISA.

14. The method of claim 12, wherein plasma is treated with a stabilizing agent subsequently to blood collection in order to enhance stability of said interleukin-18.

15. The method of claim 14, wherein said stabilizing agent is albumin.

16. The method of claim 14, wherein said stabilizing agent is a protease inhibitor.

17. The method of claim 14, wherein said stabilizing agent is a matrix metallo-protease inhibitor.

18. The method of claim 1, wherein said stem cell therapy is administration of bone marrow concentrate.

19. The method of claim 1, wherein said stem cell therapy is administration of bone marrow mononuclear cells.
